(19) United States
(12) Patent Application Publication
(10) Pub. No.: US 2025/0090792 A1
(43) Pub. Date: Mar. 20, 2025

(54) SENSORY STIMULUS PRESENTATION DEVICE, SENSORY STIMULUS PRESENTATION METHOD, AND PROGRAM

(71) Applicant: NIPPON TELEGRAPH AND TELEPHONE CORPORATION, Tokyo (JP)

(72) Inventors: Hironobu CHIBA, Tokyo (JP); Hiroaki ITO, Tokyo (JP); Kenichi NOGUCHI, Tokyo (JP); Tatsuya KAKO, Tokyo (JP)

(73) Assignee: NIPPON TELEGRAPH AND TELEPHONE CORPORATION, Tokyo (JP)

(21) Appl. No.: 18/294,500

(22) PCT Filed: Aug. 3, 2021
(86) PCT No.: PCT/JP2021/028810

§ 371 Date: Feb. 1, 2024

(51) Int. Cl. A61M 21/02; A61M 21/00

(52) U.S. Cl. CPC A61M 21/02; A61M 2021/0027

(57) ABSTRACT

The sensory stimulus presentation device includes: a label acquisition unit that acquires a classification label of an intellectual task to be performed by a user; and a sound emission unit that emits a first type of sound satisfying a predetermined criterion when the acquired classification label indicates a creativity task, and emits a second type of sound satisfying a criterion different from that of the first type of sound when the acquired classification label indicates a storage task.

### TECHNICAL FIELD

The present invention relates to a sensory stimulus presentation device, a sensory stimulus presentation method, and a program for presenting a sensory stimulus so as to obtain a desired effect.

### BACKGROUND ART

In order to promote continued concentration, there is the prior art for presenting artificial sensory stimuli such as visual, auditory, and tactile stimuli. NPL 1, for example, suggests the possibility of creating calmness in tense situations by presenting heartbeat-like tactile stimuli slower than the heartbeat.

### CITATION LIST

### Non Patent Literature

[NPL 1] Ruben T. Azevedo, Nell Bennett, Andreas Bilicki, Jack Hooper, Fotini Markopoulou and Manos Tsakiris, “The calming effect of a new wearable device during the anticipation of public speech,” Sci Rep 7, Article number: 2285, 2017.

### SUMMARY OF INVENTION

### Technical Problem

When the type or characteristics of the intellectual task executed by the user changes, there is a possibility that the sensory stimulus acting positively on the user is changed, but the prior art does not take this point into consideration.

Therefore, an object of the present invention is to provide a sensory stimulus presentation device capable of providing a sensory stimulus from an external environment so as to obtain a desired effect in consideration of the type or characteristics of an intellectual task executed by a user.

### Solution to Problem

The sensory stimulus presentation device of the present invention includes a label acquisition unit and a sound emission unit.

The label acquisition unit acquires a classification label of an intellectual task to be performed by a user. The sound emission unit emits a first type of sound satisfying a predetermined criterion when the acquired classification label indicates a creativity task, and emits a second type of sound satisfying a criterion different from that of the first type of sound when the acquired classification label indicates a storage task.

### Advantageous Effects of Invention

According to the sensory stimulus presentation device of the present invention, a sensory stimulus from an external environment can be provided so as to obtain a desired effect in consideration of the type or characteristics of an intellectual task executed by a user.

### BRIEF DESCRIPTION OF DRAWINGS

FIG. 1 is a block diagram showing a functional configuration of a sensory stimulus modulation device according to Example 1.

FIG. 2 is a flowchart showing an operation of the sensory stimulus modulation device according to Example 1.

FIG. 3 is a diagram showing an example of categorizing intellectual tasks based on a requested ability.

FIG. 4 is a diagram showing an example of quantitatively evaluating an ability required for each intellectual task.

FIG. 5 is a block diagram showing a functional configuration of a sensory stimulus presentation device according to Modification 1.

FIG. 6 is a flowchart showing an operation of the sensory stimulus presentation device according to Modification 1.

FIG. 7 is a block diagram showing a functional configuration of a sensory stimulus presentation device according to Modification 2.

FIG. 8 is a flowchart showing an operation of the sensory stimulus presentation device according to Modification 2.

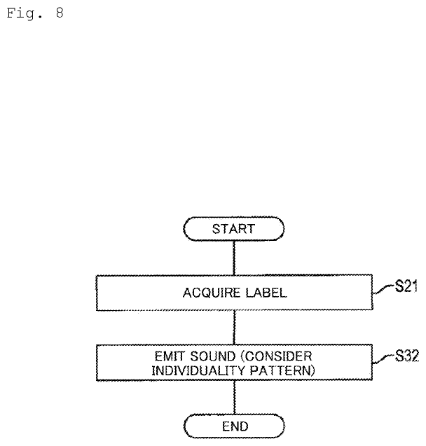

FIG. 9 is a diagram showing an example of a functional configuration of a computer.

### DESCRIPTION OF EMBODIMENTS

The following describes an embodiment of the present invention in detail. Note that components having the same function are denoted by the same number and redundant description thereof is omitted.

### Embodiment 1

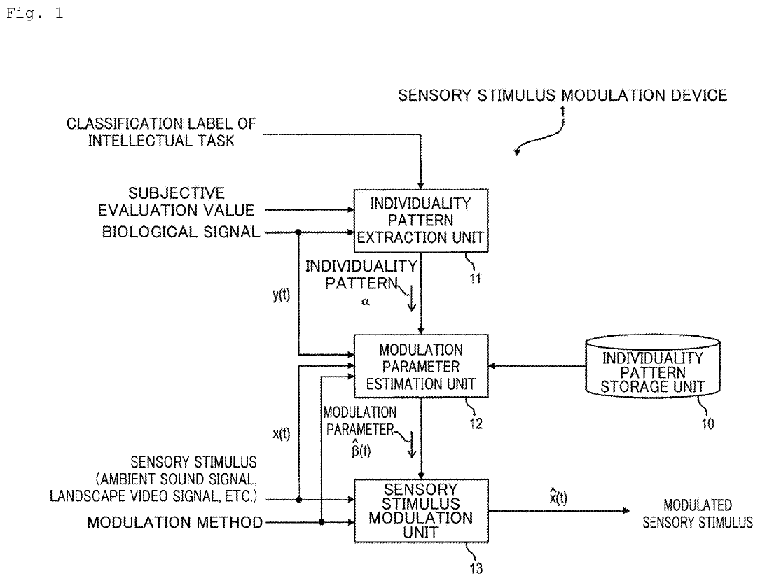

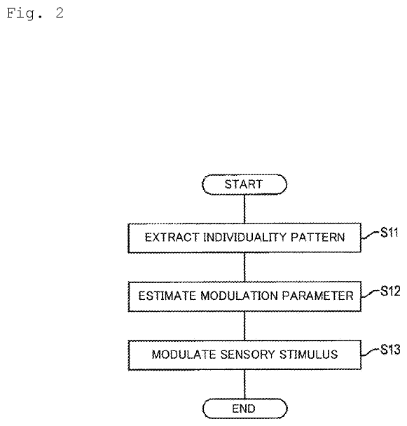

A functional configuration of a sensory stimulus modification device of Example 1 will be described with reference to FIG. 1. As shown in FIG. 1, a sensory stimulus modulation device 1 of the present example includes an individuality pattern extraction unit 11, a modulation parameter estimation unit 12, and a sensory stimulus modulation unit 13. Referring to FIG. 2, the operations of the respective configuration requirements are described below.

### <Individuality Pattern Extraction Unit 11>

Input: A subjective evaluation value, a biological signal y(t), and a classification label of an intellectual task Output: Parameter representing individuality (individuality pattern α)

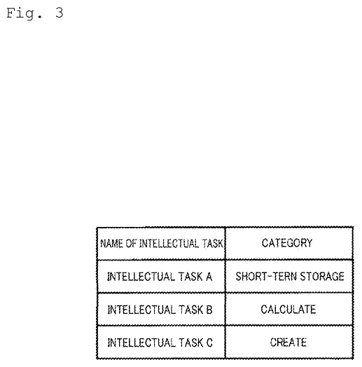

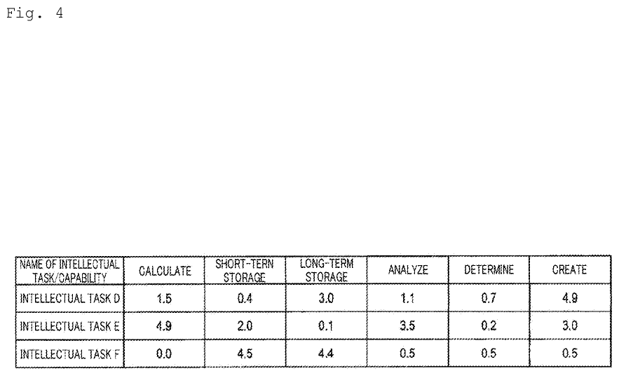

The subjective evaluation value is, for example, the answer result of a prior questionnaire. The biological signal is, for example, a brain wave signal, a heartbeat, or the like. The classification label of the intellectual task is a label indicating the type or characteristics of the intellectual task that a target user works on when using the sensory stimulus modulation device. FIGS. 3 and 4 show examples of classification of intellectual tasks. FIG. 3 is a diagram showing an example of categorizing intellectual tasks based on a required ability, and FIG. 4 is a diagram showing an example of quantitatively evaluating an ability required for each intellectual task.

Processing: The individuality pattern extraction unit 11 extracts the individuality pattern α on the basis of a predetermined criterion by inputting the subjective evaluation value, the biological signal y(t), and the classification label of the intellectual task, and outputs the individuality pattern α (S11). The predetermined criterion corresponds to, for example, the Hermann model or Plutchik model. An individuality pattern is mainly classified according to the individual questionnaire results. In addition to the criterion described above, an algorithm for classifying the individuality pattern from the brain wave signal or the like may be used.

### <Modulation Parameter Estimation Unit 12>

Input: Biological signal y(t), individuality pattern α, sensory stimulus x(t), modulation method (modulation scheme) Output Modulation parameter β\hat{ }(t) The modulation parameter estimation unit 12 estimates the modulation parameter β\hat{ }(t) for modulating the sensory stimulus x(t) to a stimulus x\hat{ }(t) optimum for increasing the concentration of the target user by using a designated modulation method by inputting the biological signal y(t), the individuality pattern α output from the individuality pattern extraction unit 11, the sensory stimulus x(t), and a modulation method (S12).

The modulation method may be set in advance in the sensory stimulus modulation device 1. The modulation method available in the present example includes, for example, increasing/reducing an amplitude value, increasing/reducing a frequency component (equalization), amplitude modulation, and mixing some signals (noise or the like) when the sensory stimulus is an acoustic signal, and increasing/reducing the luminance, increasing/reducing a frequency component (changing the hue), and mixing noise components when the sensory stimulus is a video signal. The available modulation methods are not limited to the methods described above; any method may be used as long as the sensory stimulus can be modulated to an optimal stimulus. The modulation parameter estimation unit 12 outputs the estimated modulation parameter β\hat{ }(t) to the sensory stimulus modulation unit 13.

The processing of the modulation parameter estimation unit 12 will be described in more detail. First, the modulation parameter estimation unit 12 selects a model matching the individuality pattern α output by the individuality pattern extraction unit 11, from the individuality pattern storage unit 10.

Next, the modulation parameter estimation unit 12 estimates the current concentration level from the biological signal y(t) output by the biological signal acquisition unit 11. The modulation parameter estimation unit 12 successively estimates the optimum stimulus x\hat{ }(t) so as to further increase the concentration level by assuming that the current concentration level corresponds to the current stimulus x(t) in the selected model. Finally, in the designated modulation method, the modulation parameter β\hat{ }(t) required to obtain the optimum stimulus x\hat{ }(t) from the current stimulus x(t) is successively estimated. That is, β\hat{ }(t) satisfying x\hat{ }(t)=h (x(t), β\hat{ }(t)) is estimated. Here, h(⋅) is a function for changing the stimulus x(t) to the stimulus x\hat{ }(t) by using the parameter β\hat{ }(t), and a specific function is determined according to the designated modulation method.

The following table lists functions corresponding to the available modulation schemes and parameters required for each function. This list is merely an example, and various modulation schemes and parameters that can modulate external stimuli such as acoustic and video signals can be used.

TABLE 1

| Modulation method (modulation scheme) | Necessary parameters |
|---|---|
| Increasing/reducing amplitude x\hat{ }(t) = V _{β} x(t) | V _{β} |
| AM modulation x\hat{ }(t) = (V _{β} cosω _{β}t) · x(t) | V _{β}, ω _{β} |

### <Sensory Stimulus Modulation Unit 13>

Input Sensory Stimulus x(t), Modulation Method, Modulation Parameter β\hat{ }(t)
Output Modulated Sensory Stimulus x\hat{ }(t)

Processing: The sensory stimulus modulation unit 13 inputs the sensory stimulus x(t), a modulation method (modulation scheme), and the modulation parameter β\hat{ }(t), to obtain the sensory stimulus x\hat{ }(t) obtained by modulating the sensory stimulus x(t) using the modulation parameter β\hat{ }(t) on the basis of the designated modulation scheme (S13). The sensory stimulus modulation unit 13 presents the modulated sensory stimulus x\hat{ }(t) so that the target user can perceive it.

In a case where the sensory stimulus x(t) is an ambient sound signal, a sound based on the modulated sensory stimulus x\hat{ }(t) is output from a sound emission unit (not shown) such as a speaker and a headphone that is set at a position and a sound volume at which the target user can listen. Examples of the emitted sensory stimulus x\hat{ }(t) include a sensory stimulus by binaural beat (Reference NPL 1).
(Reference NPL 1: J. Park, H. Kwon, S. Kang and Y. Lee, “The effect of binaural beatbased audiovisual stimulation on brain waves and concentration,” 2018 International Conference on Information and Communication Technology Convergence (ICTC), pp. 420-423, 2018.)

When the sensory stimulus x(t) is a landscape video signal, a video based on the modulated sensory stimulus x\hat{ }(t) is output from a display unit (not shown) such as a display or head-mounted display installed at a position where the target user can view.

### Effects

The sensory stimulus modulation device 1 of the present example performs processing in which the type of an intellectual task is added to the individuality pattern of the target user. As a result, even in the combination of the user and the type of the intellectual task, for which the effect of continued/improved concentration is low in the conventional method, it is expected that the interruption of the concentration state is reduced and the concentration power is improved.

In addition, unlike the prior art, the sensory stimulus modulation device 1 of the present example can add the type or characteristics of the intellectual task that the target user works on in accordance with the individuality pattern (model representing the relationship between the concentration level and the sensory stimulus) of the target user.

### Evaluation Experiment

An evaluation experiment was carried out on the relationship between the performance of an intellectual task and ambient sound. This evaluation experiment is an experiment in which a subject is asked to engage in two types of intellectual tasks (creativity task, word memory task) while listening to one of eight types of ambient environment sounds (background noise, vacuum cleaner noise, ambient sound in a restaurant, ambient sound in a train, ambient sound in an office, voices of children, sound of a murmuring river, music (jazz)), with the aim of verifying whether or not there are changes in task performance/feeling depending on the type of ambient sound.

As a result of the experiment, in the creativity task, work performance was improved by listening to the ambient sound (ambient sound in a train, ambient sound in a restaurant, ambient sound in an office, vacuum cleaner noise, or the like) that occurs indoor in everyday life, while listening to the voices of children, background noise, and music has dropped the work performance.

On the other hand, in the word storage task, there is a tendency different from that in the creativity task. In the word storage task, by listening to an ambient environmental sound (background noise, sound of a murmuring river, ambient sound in a train, ambient sound of an office, etc.) having little temporal variation of timbre, the work performance has improved. On the other hand, listening to the ambient environmental sound (ambient sound in a restaurant, music, voices of children, etc.) having a large temporal variation of timbre has dropped the work performance.

### Modification 1

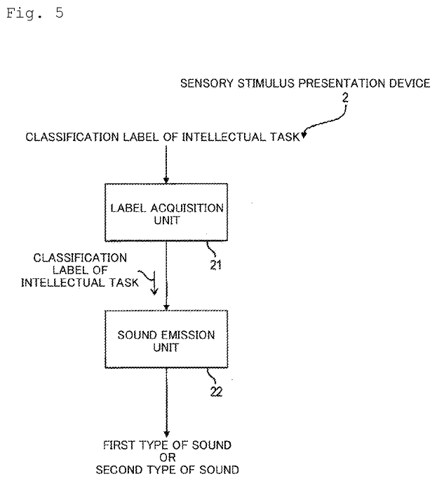

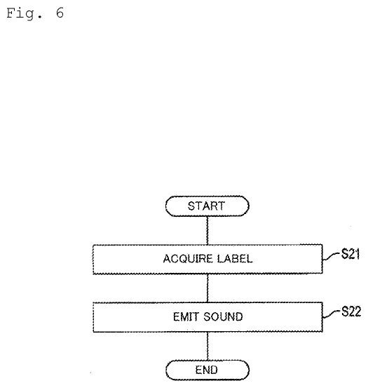

Based on the results of the evaluation experiment described above, the sensory stimulus presentation device 2 of Modification 1 shown in FIG. 5 is disclosed. As shown in the drawing, the sensory stimulus presentation device 2 includes a label acquisition unit 21 and a sound emission unit 22. An operation of the sensory stimulus presentation device 2 of the present modification will be described with reference to FIG. 6.

### <Label Acquisition Unit 21>

Input: Classification label of intellectual task Output Classification label of intellectual task

Processing: The label acquisition unit 21 acquires a classification label of an intellectual task to be performed by a user (S21).

### <Sound Emission Unit 22>

Input: Classification label of intellectual task Output First type of sound or second type of sound

Processing: The sound emission unit 22 emits the first type of sound satisfying a predetermined criterion when the acquired classification label indicates the creativity task, and emits the second type of sound satisfying a criterion different from that of the first type of sound when the acquired classification label indicates the storage task (S22). The first type of sound is preferably selected as a sound that satisfies the criterion based on ambient sound generated in a room in everyday life (e.g., ambient sound in a car of a train, ambient sound in a restaurant, ambient sound in an office, vacuum cleaner noise, etc.) The second type of sound is preferably selected as a sound that satisfies the criterion based on an ambient environmental sound (e.g., background noise, sound of a murmuring river, ambient sound in a car of a train, ambient sound in an office, and the like described above) with little temporal variation of timbre.

### Modification 2

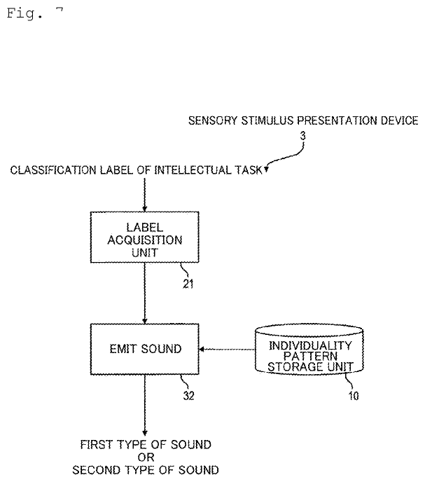

The individuality pattern storage unit 10 for storing in advance an individuality pattern as a parameter representing the individuality may be added to the sensory stimulus presentation device 2 of Modification 1. Specifically, as shown in FIG. 7, a sensory stimulus presentation device 3 of Modification 2 includes the label acquisition unit 21, the sound emission unit 32, and the individuality pattern storage unit 10, wherein the sound emission unit 32 modulates the first type of sound for a corresponding user or the second type of sound for a corresponding user on the basis of the individuality pattern stored in the individuality pattern storage unit 10 and emits the sound (S32).

### Additional Note

The device according to the present invention is constituted as, for example, a single hardware entity that includes an input unit to which a keyboard or the like can be connected, an output unit to which a liquid crystal display or the like can be connected, a communication unit to which a communication device (e.g., communication cable) that enables communication with the outside of the hardware entity can be connected, a CPU (Central Processing Unit, which may include a cash memory, a register, etc.), RAM and ROM, which are memories, an external storage device that is a hard disk, and a bus that connects the input unit, the output unit, the communication unit, the CPU, the RAM, the ROM, and the external storage device to enable data exchange therebetween. Also, if necessary, the hardware entity may be provided with a device (drive) or the like capable of reading and writing data from/to a recording medium such as a CD-ROM. An example of a physical entity including such hardware resources is a general-purpose computer.

Programs required for realization of the above-described functions, data required for processing of the programs, and the like are stored in the external storage device of the hardware entity (the present invention is not limited to an external storage device, and for example, the programs may be read out and stored in a ROM, which is a dedicated storage device). Further, data and the like obtained by the processing of the programs is appropriately stored in a RAM, an external storage device, or the like.

In the hardware entity, each program stored in the external storage device (or ROM, etc.) and data necessary for the processing of each program are read into a memory as necessary, and interpreted, executed, and processed by the CPU as appropriate. As a result, the CPU implements predetermined functions (each of the constituent requirements described above as XXX unit, XXX means, etc.).

The present invention is not limited to the above-described embodiment, and appropriate changes can be made without departing from the spirit of the present invention. Further, the processes described in the embodiments are not only executed in time series in the described order, but also may be executed in parallel or individually according to a processing capability of a device that executes the processes or as necessary.

As described above, when a processing function in the hardware entity (the device of the present invention) described in the above embodiment is realized by a computer, processing content of functions that the hardware entity needs to have is described by a program. A processing function in the above hardware entity is realized on the computer by executing this program on the computer.

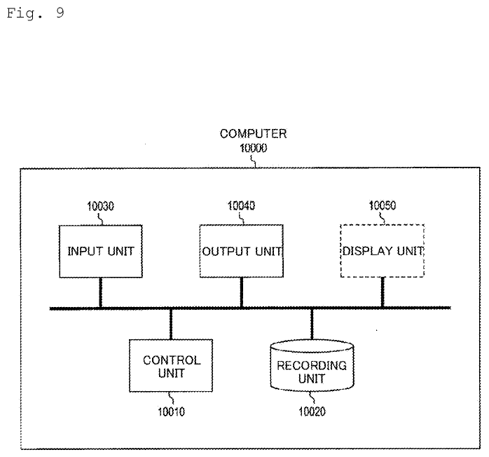

The above-described various types of processing can be performed by reading a program executing each step of the foregoing methods to a storage unit 10020 of a computer 10000 illustrated in FIG. 9 and operating a control unit 10010, an input unit 10030, an output unit 10040, or the like.

A program describing this processing content can be recorded on a computer-readable recording medium. An example of the computer-readable recording medium may include any recording medium such as a magnetic recording device, an optical disc, a magneto-optical recording medium, and a semiconductor memory. Specifically, for example, a hard disk device, a flexible disk, magnetic tape, or the like can be used as the magnetic recording device, a DVD (Digital Versatile Disc), a DVD-RAM (Random Access Memory), a CD-ROM (Compact Disc Read Only Memory), a CD-R (Recordable)/RW (Rewritable), or the like can be used as the optical disk, an MO (Magneto-Optical disc) or the like can be used as the magnetooptical recording medium, and an EEP-ROM (Electrically Erasable and Programmable-Read Only Memory) or the like can be used as the semiconductor memory.

In addition, distribution of this program is carried out by, for example, selling, transferring, lending, or the like a portable recording medium such as a DVD or a CD-ROM on which the program is recorded. Further, the program may be distributed by being stored in a storage device of a server computer and transferred from the server computer to another computer via a network.

The computer that executes such a program first temporarily stores, for example, the program recorded on the portable recording medium or the program transferred from the server computer in a storage device of the computer. Furthermore, when processing is performed, the computer reads the program stored in the own recording medium and performs the processing in accordance with the read program. In addition, as another execution mode of the program, a computer may directly read the program from a portable recording medium and execute processing in accordance with the program. Further, whenever the program is transmitted from the server computer to the computer, the processing may be performed in order in accordance with the received program. Furthermore, instead of transferring the program to the computer from a server computer, the processing described above may be executed by a so-called ASP (Application Service Provider) type service, in which a processing function is realized by execution commands and result acquisition alone. Note that the program in this embodiment includes information to be used for processing by a computer and equivalent to the program (data which is not a direct command to the computer but has a property that regulates the processing of the computer and the like).

Further, although the hardware entity is configured by a predetermined program being executed on the computer in the present embodiment, at least a part of the processing content of the hardware entity may be realized in hardware.

1. A sensory stimulus presentation device comprising a processor configured to execute operations comprising:
acquiring a classification label of an intellectual task to be performed by a user;
emitting a first type of sound satisfying a predetermined criterion when the acquired classification label indicates a creativity task; and
emitting a second type of sound satisfying a criterion different from that of the first type of sound when the acquired classification label indicates a storage task.

2. The sensory stimulus presentation device according to claim 1,
wherein the first type of sound is a sound satisfying a criterion based on an ambient sound normally generated in a room.

3. The sensory stimulus presentation device according to claim 2,
wherein the first type of sound is at least one of an ambient sound in a car of a train, an ambient sound in a restaurant, an ambient sound in an office, or a vacuum cleaner noise.

4. The sensory stimulus presentation device according to claim 1,
wherein the second type of sound satisfies a criterion based on an ambient environmental sound having temporal variation in timbre within a predetermined threshold.

5. The sensory stimulus presentation device according to claim 4,
wherein the second type of sound is at least one of a background noise, a sound of a murmuring river, an ambient sound in a car of a train, and an ambient sound in an office.

6. The sensory stimulus presentation device according to claim 1,
the processor further configured to execute operations comprising:
modulating either the first type of sound for a corresponding user or the second type of sound for a corresponding user on the basis of an individuality pattern of the user; and
emitting the modulated sound.

7. A computer implemented method for presenting a sensory stimulus, the method comprising:
acquiring a classification label of an intellectual task to be performed by a user;
emitting a first type of sound satisfying a predetermined criterion when the acquired classification label indicates a creativity task; and
emitting a second type of sound satisfying a criterion different from that of the first type of sound when the acquired classification label indicates a storage task.

8. A computer-readable non-transitory recording medium storing a computer-executable program instructions that when executed by a processor cause a computer system to execute operations comprising:
acquiring a classification label of an intellectual task to be performed by a user;
emitting a first type of sound satisfying a predetermined criterion when the acquired classification label indicates a creativity task; and
emitting a second type of sound satisfying a criterion different from that of the first type of sound when the acquired classification label indicates a storage task.

9. The sensory stimulus presentation device according to claim 2,
the processor further configured to execute operations comprising:
modulating either the first type of sound for a corresponding user or the second type of sound for a corresponding user on the basis of an individuality pattern of the user; and
emitting the modulated sound.

10. The computer implemented method according to claim 7,
wherein the first type of sound is a sound satisfying a criterion based on an ambient sound normally generated in a room.

11. The computer implemented method according to claim 7,
wherein the first type of sound is at least one of an ambient sound in a car of a train, an ambient sound in a restaurant, an ambient sound in an office, or a vacuum cleaner noise.

12. The computer implemented method according to claim 7,
wherein the second type of sound satisfies a criterion based on an ambient environmental sound having temporal variation in timbre within a predetermined threshold.

13. The computer implemented method according to claim 12,
wherein the second type of sound is at least one of a background noise, a sound of a murmuring river, an ambient sound in a car of a train, and an ambient sound in an office.

14. The computer implemented method according to claim 7, further comprising:
modulating either the first type of sound for a corresponding user or the second type of sound for a corresponding user on the basis of an individuality pattern of the user; and
emitting the modulated sound.

15. The computer-readable non-transitory recording medium according to claim 8, wherein the first type of sound is a sound satisfying a criterion based on an ambient sound normally generated in a room.

16. The computer-readable non-transitory recording medium according to claim 8, wherein the first type of sound is at least one of an ambient sound in a car of a train, an ambient sound in a restaurant, an ambient sound in an office, or a vacuum cleaner noise.

17. The computer-readable non-transitory recording medium according to claim 8, wherein the second type of sound satisfies a criterion based on an ambient environmental sound having temporal variation in timbre within a predetermined threshold.

18. The computer-readable non-transitory recording medium according to claim 17, wherein the second type of sound is at least one of a background noise, a sound of a murmuring river, an ambient sound in a car of a train, and an ambient sound in an office.

19. The computer-readable non-transitory recording medium according to claim 8, the computer-executable program instructions when executed further causing the computer system to execute operations comprising:
modulating either the first type of sound for a corresponding user or the second type of sound for a corresponding user on the basis of an individuality pattern of the user; and
emitting the modulated sound.

20. The computer-readable non-transitory recording medium according to claim 15, the computer-executable program instructions when executed further causing the computer system to execute operations comprising:
modulating either the first type of sound for a corresponding user or the second type of sound for a corresponding user on the basis of an individuality pattern of the user; and
emitting the modulated sound.
